(19) United States
(12) Patent Application Publication
(10) Pub. No.: US 2015/0342123 A1
(43) Pub. Date: Dec. 3, 2015

(54) METHOD FOR COLORING FLOWERS ON A PLANT AND A PLANT WITH COLORED FLOWERS

(71) Applicant: Robert McCurdy, Sorrento, FL (US)

(72) Inventor: Robert McCurdy, Sorrento, FL (US)

(21) Appl. No.: 14/288,871

(22) Filed: May 28, 2014

(51) Int. Cl. A01G 1/00; A01H 5/02

(52) U.S. Cl. CPC A01G 1/001; A01H 5/02

(57) ABSTRACT

A method for coloring flowers on a plant includes the steps of identifying a suitable plant and selecting one or more flowers on the plant to receive a color enhancement; air layering a portion of the plant on the stem and/or a stem branch; selecting a dye having a color and/or a fragrance and securing the same against the air layered portion of the plant. A plant with colored flowers includes a plant that has undergone the above noted steps.

### TECHNICAL FIELD

The present invention relates generally to a method for changing the color of a flower, and more particularly to a plant with a colored portion and a method for making the same.

### BACKGROUND

The statements in this section merely provide background information related to the present disclosure and may not constitute prior art.

Orchids are among the most beautiful and desirable plants available for purchase in the United States and around the world. For centuries, orchids have been coveted for their beauty, fragrance and mythical healing powers. Moreover, orchids are prized for their long lasting blooms which are known to remain open and beautiful for months at a time.

Although there are some forms of natural orchids which include colored blooms, the vast majority of commercially grown orchids are white. In this regard, and owing to the popularity of orchids in general, recent attempts have been made to introduce orchids having colors other than white.

For example, U.S. Patent Publication Nos. 2011/0252511, and 2013/0239474, are directed to methods for coloring orchids wherein a large cavity is bored into the delicate stem of an orchid, and a pipette is used to fill the cavity with a large amount of dye. This dye is immediately absorbed into the plant and carried to the flowers emanating from the plant stem.

Although these methods are useful for simply introducing color to the flower portion of an orchid, they suffer from many practical drawbacks. For example, by creating a cavity within the stem that is large enough to receive all of the dye, the stability of the plant itself is severely weakened. As such, many of these colored orchids are so fragile that the stem breaks at the injection site during transport to merchants for sale and/or when being handled by an end user. Moreover, owing to the requirement for an opening large enough to receive all of the dye, the main stem of the orchid must be injected. As a result, all of the flowers on the orchid receive the same dye, and cannot include different colors from another flower on the same plant.

Additionally, because all of the dye is injected directly into the plant at one time, the plant can go into shock, resulting in a much shorter lifespan. In this regard, it is not uncommon for orchids that have been subjected to the above methods to wither and die within 30 days from being injected, whereas traditional non-dyed orchids will produce blooms that last between three and six months. Moreover, because the orchids are bombarded with dye, it is not uncommon for the flowers to lose their trademarked scent. As such, many consumers note that orchids which have been colored as described above tend to smell like the chemical dyes which were introduced to produce the colored flowers.

Accordingly, there remains a need for improved methods for coloring flowers on a plant, and the resulting plants, which do not suffer from the drawbacks of the above noted art.

### SUMMARY OF THE INVENTION

The present invention is directed to a method for coloring flowers on a plant and a plant with colored flowers.

One embodiment of the present invention can include a method for coloring flowers on a plant wherein the plant itself is not damaged and the lifespan of the plant is not negatively affected by the coloring procedure. The method can include several steps such as identifying a suitable plant and selecting one or more flowers on the plant to receive a color enhancement. Once selected, the method can proceed to the step of air layering a portion of the plant on the stem and/or a stem branch. Next, a dye having a color and/or a fragrance can be placed against the air layered portion of the plant, in order to allow the selected flower(s) to receive the color and/or fragrance from the dye.

Another embodiment of the present invention can include a plant with colored flowers that has undergone the above noted method.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1 illustrates one embodiment of a plant with colored flowers that is useful for understanding the inventive concepts disclosed herein.

FIG. 2 is a process flowchart detailing a method for coloring the flowers of a plant, in accordance with one embodiment of the invention.

FIG. 3 is a detail perspective view of a plant stem, in accordance with one embodiment of the invention.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

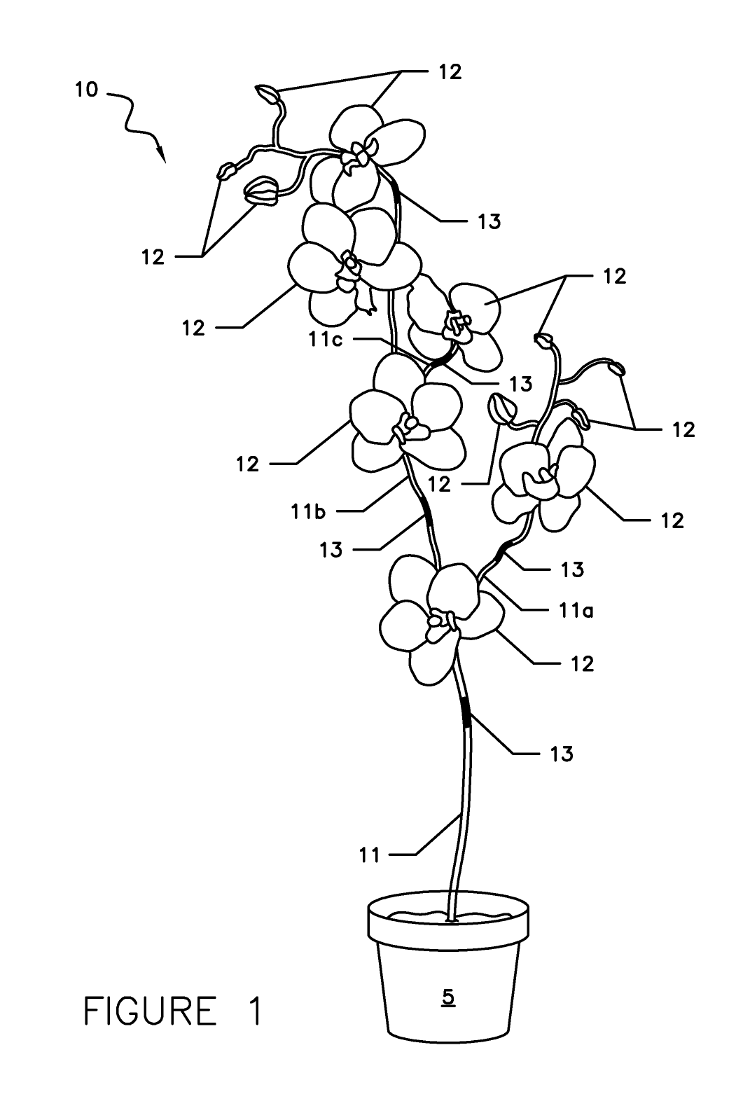

Identical reference numerals are used for like elements of the invention or elements of like function. For the sake of clarity, only those reference numerals are shown in the individual figures which are necessary for the description of the respective figure. For purposes of this description, the terms “upper,” “bottom,” “right,” “left,” “front,” “vertical,” “horizontal,” and derivatives thereof shall relate to the invention as oriented in FIG. 1. As described herein, the term “flower” and derivatives thereof shall include both a flower bud and bloom. As described herein, the term “stem branch” and derivatives thereof can refer to the portion of the plant from which the flowers are connected. Although illustrated as including separate elements extending outward from the main stem, other embodiments are contemplated wherein the main stem gradually tapers down in size and is connected to the flowers.

FIGS. 1-3 illustrate one embodiment of a method for coloring flowers on a plant, and a plant with colored flowers. Although the below described methodology is illustrated with respect to a white phalaenopsis, this is for illustrative purposes only, as the inventive concepts disclosed herein can function to provide color and/or fragrance to the flower(s) of any number of different types and species of plant life. As such, the invention is not to be construed as limiting only to orchids.

FIG. 1 illustrates one embodiment of a plant with colored flowers 10. As shown, the plant can include a main stem 11, one or more stem branches 11a, 11b and 11c, for example, and a plurality of flowers 12. Although not illustrated, one or more support rods can be positioned against the stem(s) in order to direct the growth of the plant upward from a nursery pot 5 or other such vessel. The plant 10 can also include one or more air layered sections 13 for receiving a dye and/or fragrance.

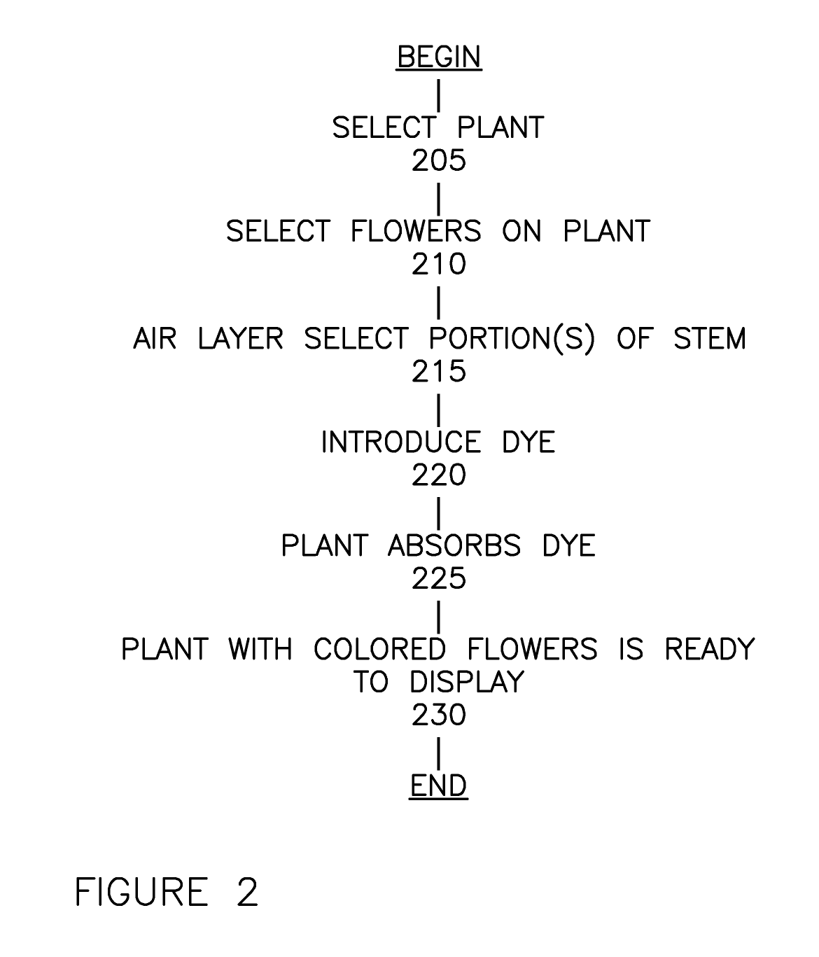

FIG. 2 is a flow chart illustrating a method 200 for coloring flowers on a plant. The method can begin at step 205, wherein a suitable plant is selected. In the preferred embodiment, the plant can comprise a white phalaenopsis (i.e., orchid), however any number of other suitable plants are also contemplated.

Once a suitable plant has been identified, the method can proceed to step 210, wherein a user can select which flowers on the plant are to receive a color enhancement. In this regard, and as will become apparent from the following description, the user can allow each of the plant's flowers to receive an identical color and fragrance, or can allow one or more of the plant's flowers to receive a different color and/or fragrance.

Once the selection of flower(s) has been made, the method can proceed to step 215, wherein a portion of the stem and/or stem branch that is located between each selected flower and the root can be air layered. As described herein, the term “air layered” and derivatives thereof can include a process by which a portion of the outermost surface (i.e., bark) of the main stem 11 and/or one or more stem branches 11a, 11b and 11c can be scraped and/or partially removed so as to expose the moist interior portion of the stem, i.e., the xylem. In this regard, it is specifically noted that the xylem is neither cut, pierced or otherwise damaged by this process, so as to ensure the structure/stability of the plant itself is not weakened.

Next, the method can proceed to step 220 wherein a dye can be selected and then introduced onto the air layered section(s) of the plant.

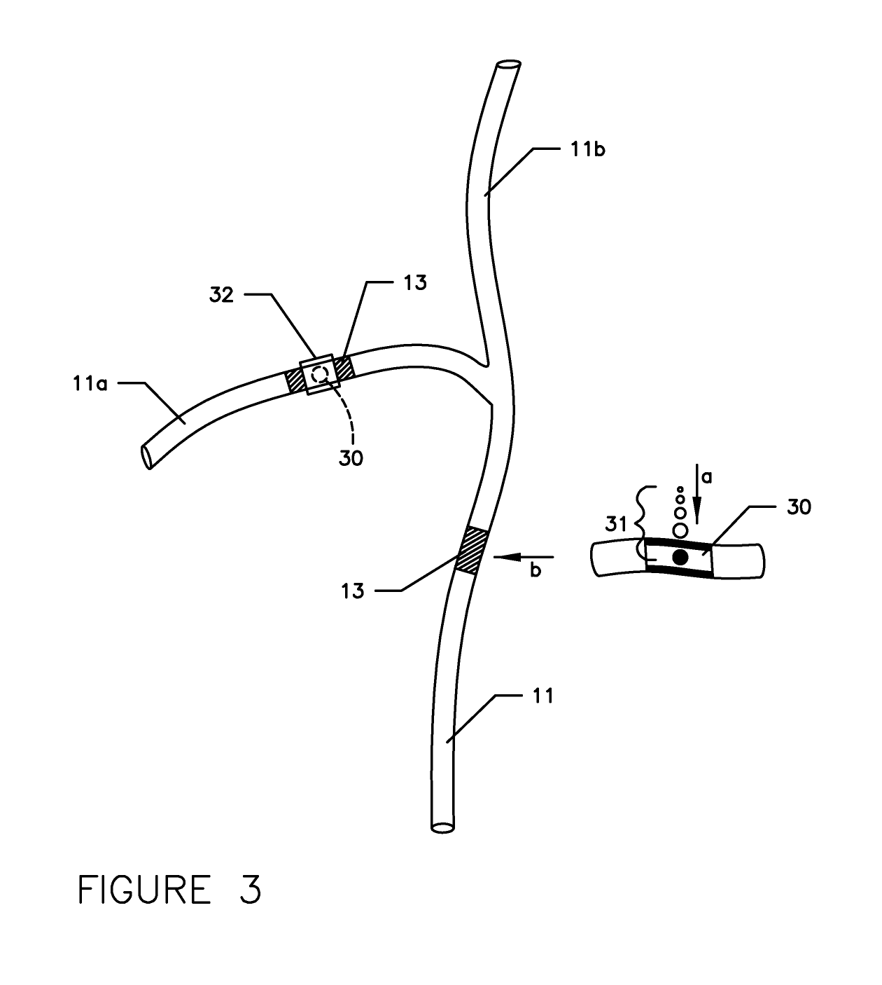

FIG. 3 illustrates one preferred embodiment of introducing dye onto the air layered section of the plant. As shown, an absorbent material 30 such as a cotton ball, for example, can be saturated with a liquid and/or solid dye 31 (see arrow a) having any number of different colors and/or fragrances. The absorbent material 30 can be secured (see arrow b) onto the air layered section of the plant via a connector 32 such as a piece of tape or a clip, for example, in order to allow the dye to be gradually introduced into the plant stem. Of course, any number of other different absorbent materials, dyes and/or connectors can also be utilized herein.

Next, the method can proceed to step 225, wherein a desired portion of the dye 31 is absorbed by the plant. As such, the plant can be watered and the dye 31 can be absorbed by the rising sap that flows within the plant stem until it is delivered to the flower. In this regard, the absorbent material can remain affixed to the plant stem for any desirable length of time. As described herein, the term “desired” and derivatives thereof can include a scenario in which the absorbent material remains affixed to the plant until all of the dye has been introduced into the plant stem, and/or until the one or more of the petals in each flower takes on a color and/or scent that is acceptable to the user. For example, the longer the dye remains in contact with the plant, the more vibrant the color of the flower will be. In either instance, once the desired look has been achieved the absorbent material 30 can be removed.

As described herein, any number of commercially available nontoxic dyes can be used to impart color into the flower. Likewise, any number of commercially available scented oils in an unlimited number of different fragrances can also be utilized.

Finally, the method can proceed to step 230, wherein the plant with colored flowers is ready for display and/or sale to a consumer.

As described herein, the method 200 for coloring flowers on a plant can allow a user to produce a plant 10 having any number of different colored flowers 12. Moreover, by providing multiple air layers 13 and 13′ along a single stem branch (e.g., 11a), the separate colors and/or fragrances introduced into these layers can combine to form a unique color and/or fragrance within those flowers located distal to the air layers.

Moreover, owing to the fact that the inside portion of the plant stems are not damaged, and that the dye 31 is gradually absorbed by the plant, the above described plant with colored flowers are no less fragile than non-colored plants and enjoy no reduction lifecycle.

As to a further description of the manner and use of the present invention, the same should be apparent from the above description. Accordingly, no further discussion relating to the manner of usage and operation will be provided.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A method for coloring flowers on a plant, said method comprising:
selecting a plant having a main stem that is in communication with one or more flowers;
performing an air layering procedure on a portion of the main stem;
selecting a dye, and depositing the same onto an absorbent material;
securing, via a connector, the absorbent material and dye onto the air layered portion of the main stem; and
removing the absorbent material after a desired portion of the dye has been absorbed by the plant.

2. The method of claim 1, wherein the air layering procedure includes at least one of the step of scraping a portion of the outermost surface of the stem to expose the xylem, and the step of removing a portion of the outermost surface of the stem to expose the xylem.

3. The method of claim 1, wherein selecting a dye includes the steps of selecting a color, and selecting a fragrance.

4. The method of claim 1, wherein the connector includes at least one of a clip and an adhesive material.

5. A plant having one or more flowers that are colored by means of the method of claim 1.

6. The method of claim 1, wherein said plant further includes at least one stem branch, and the method further comprises:
performing an air layering procedure on a portion of the at least one stem branch;
selecting another dye, and depositing the same onto another absorbent material;
securing, via another connector, the another absorbent material and another dye onto the air layered portion of the stem branch; and
removing the another absorbent material after a desired portion of the another dye has been absorbed by the plant.

7. A plant having one or more flowers that are colored by means of the method of claim 6.

8. A method for coloring flowers on a plant, said method comprising:
selecting a plant having a main stem that is in communication with one or more stem branches and one or more flowers;
selecting at least one of the one or more flowers to receive a color enhancement;
performing an air layering procedure on a portion of each of the one or more stem branches that are in direct communication with each of the selected one or more flowers;
selecting a dye, and depositing the same onto at least one absorbent material;
securing, via a connector, each of the at least one absorbent material and dye onto the air layered section of each of the one or more stem branches; and
removing the each of the at least absorbent material.

9. A plant having one or more flowers that are colored by means of the method of claim 8.

10. The method of claim 8, wherein a color of the dye on each of the absorbent materials is identical.

11. The method of claim 8, wherein a color of the dye on one of the absorbent materials is different than a color of the dye on another of the absorbent materials.

12. A plant having one or more flowers that are colored by means of the method of claim 11.

13. The method of claim 8, wherein a fragrance of the dye on each of the absorbent materials is identical.

14. The method of claim 8, wherein a fragrance of the dye on one of the absorbent materials is different than a fragrance of the dye on another of the absorbent materials.

15. A method for coloring flowers on a plant, said method comprising:
selecting a plant having a main stem that is in communication with a plurality of stem branches and a plurality of flowers;
performing an air layering procedure on a portion of each of the plurality of stem branches;
selecting one or more dye, and depositing the same onto a plurality of absorbent materials;
securing, via a connector, each of the plurality of absorbent materials and dye onto the air layered sections of each of the plurality of stem branches; and
removing each of the plurality of absorbent materials.

16. A plant having one or more flowers that are colored by means of the method of claim 15.

17. The method of claim 15 wherein a color of the dye on one of the absorbent materials is different than a color of the dye on another of the absorbent materials.

18. A plant having one or more flowers that are colored by means of the method of claim 17.

19. The method of claim 15, wherein a fragrance of the dye on each of the absorbent materials is identical.

20. The method of claim 1, further comprising:
performing an air layering procedure on a portion of the main stem;
selecting another dye, and depositing the same onto another absorbent material;
securing, via another connector, the another absorbent material and another dye onto the air layered portion of the main stem; and
removing the another absorbent material after a desired portion of the another dye has been absorbed by the plant.
